(19) United States
(12) Patent Application Publication
(10) Pub. No.: US 2012/0265114 A1
(43) Pub. Date: Oct. 18, 2012

(54) STRETCHER AND METHOD OF STRETCHING LOWER BODY

(76) Inventor: Abraham A. Kang, Fairfax, VA (US)

(21) Appl. No.: 13/444,539

(22) Filed: Apr. 11, 2012

Related U.S. Application Data
(60) Provisional application No. 61/475,043, filed on Apr. 13, 2011.

(51) Int. Cl. A61F 5/00

(52) U.S. Cl. 602/35; 602/36

(57) ABSTRACT

A stretcher may stretch a lower body of a user laying down in a state in which the stretcher is arranged adjacent to, arranged on, or integrally attached to a bed. The stretcher includes an upper body movement restraint configured to prevent an upper body from being moved in the direction of stretching, a friction reducer configured to reduce a friction between the lower body and a lower body contact surface, and a tension generator configured to give a stretching force to the lower body when the upper body is held by the upper body restraint with the lower body arranged on the friction reducer. A user may be able remain flat on his or her back after using the stretcher. This may allow the user's joints to rest and/or heal in a relaxed state.

### CROSS-REFERENCE TO RELATED APPLICATIONS

This application claims priority under 35 U.S.C. §119 to U.S. Provisional Application No. 61/475,043 filed on Apr. 13, 2011, the disclosure of which is incorporated herein by reference in its entirety.

### BACKGROUND OF THE INVENTION

The present disclosure relates to an exemplary stretcher arranged adjacent to, arranged on, or mounted on a bed. The exemplary stretcher may stretch a lower body, including, for example, a spine, waist joints, and knee joints.

### SUMMARY OF THE INVENTION

The present disclosure relates to an exemplary stretcher arranged adjacent to, arranged on, or mounted on a bed. The exemplary stretcher may stretch a lower body, including, for example, a spine, waist joints, and knee joints.

In one embodiment, an exemplary stretcher for stretching a lower body of a user comprises: a base including a first section configured to hold an upper body of the user and a second section adjacent the first section, the second section configured to hold the lower body of the user, the length of the base from a head end to a foot end being L1, wherein the first section is configured to provide a first amount of friction F1 to the body of the user and the second section is configured to provide a second amount of friction F2 to the body of the user, where F2 is less than F1; an upper body movement restraint disposed at the head end of the base, the upper body movement restraint configured to prevent the upper body of the user from being moved in a longitudinal direction away from the head end of the base when a user is disposed in the stretcher; and a tension generator disposed at the foot end of the base, the tension generator comprising: a cord guide attached at the foot end of the base; at least a first lower body attachment connected via a cord to a cord hole disposed in a front surface of the cord guide, a height between an upper surface of the base and the cord hole being H1, wherein a first end of the cord is attached to the first lower body attachment, the cord extending from the first lower body attachment to the cord hole and through an inside of the cord guide, wherein the tension generator is configured to generate a stretching force via the cord to the lower body of the user when a portion of the upper body of the user is disposed in the upper body movement restraint, the lower body of the user is disposed on the second section, and a portion of the lower body of the user is connected to the first lower body attachment, and wherein the height H1 is 5%-25% of the base length L1.

In one embodiment, an exemplary stretcher for stretching a lower body of a user comprises: a base including a first section configured to hold an upper body of the user and a second section adjacent the first section, the second section configured to hold the lower body of the user, the length of the base from a head end to a foot end being L1, wherein the first section is configured to provide a first amount of friction F1 to the body of the user and the second section is configured to provide a second amount of friction F2 to the body of the user, where F2 is less than F1; an upper body movement restraint disposed at the head end of the base, the upper body movement restraint configured to prevent the upper body of the user from being moved in a longitudinal direction away from the head end of the base when a user is disposed in the stretcher; and a tension generator disposed at the foot end of the base, the tension generator comprising: a roller support adjacent the foot end of the base that protrudes upwardly; and a roller rotatable about an axis thereof, the roller attached at a top end of the roller support, wherein the tension generator is configured to cause a stretching force via the roller to the lower body of the user when a portion of the upper body of the user is disposed in the upper body movement restraint, the lower body of the user is disposed on the second portion, and a portion of the lower body of the user is disposed on the roller.

In one embodiment, an exemplary stretcher for stretching a lower body of a user comprises: a base including a first section configured to hold an upper body of the user and a second section adjacent the first section, the second section configured to hold the lower body of the user, wherein the first section is configured to provide a first amount of friction F1 to the body of the user and the second section is configured to provide a second amount of friction F2 to the body of the user, where F2 is less than F1; an upper body movement restraint disposed at the head end of the base, the upper body movement restraint configured to prevent the upper body of the user from being moved in a longitudinal direction away from the head end of the base when a user is disposed in the stretcher; and a tension generator disposed at the foot end of the base, the tension generator comprising: a cord guide attached at the foot end of the base; at least a first lower body attachment connected via a cord to a cord hole disposed in a front surface of the cord guide, wherein a first end of the cord is attached to the first lower body attachment, the cord extending from the first lower body attachment to the cord hole and through an inside of the cord guide; wherein the tension generator is configured to generate a stretching force via the cord to the lower body of the user when a portion of the upper body of the user is disposed in the upper body movement restraint, the lower body of the user is disposed on the second section, and a portion of the lower body of the user is connected to the first lower body attachment, and wherein the stretcher is integral with a bed.

### BRIEF DESCRIPTION OF THE DRAWINGS

The above and other aspects and features of the disclosure will become apparent by describing in detail exemplary embodiments thereof with reference to the attached drawings, in which:

FIG. 1 is an entire perspective view showing a schematic structure of an exemplary stretcher according to a first embodiment.

FIG. 2 is a cross-sectional view taken along the line II-II in FIG. 2

FIG. 3 is a perspective view showing an exemplary upper body restraint as a part of a stretching force giving mechanism of the stretcher according to the first embodiment.

FIG. 4 is a perspective view showing an exemplary ankle strap as a part of a stretching force giving mechanism of the stretcher according to the first embodiment.

FIG. 5 is a perspective view showing an exemplary friction reducing member of the stretcher according to the first embodiment.

FIG. 6 is an entire perspective view showing an exemplary stretcher according to a second embodiment.

FIG. 7 is a cross-sectional view taken along the line VII-VII in FIG. 6.

FIG. 8 is an entire perspective view showing an exemplary stretcher according to a third embodiment.

FIG. 9 is a cross-sectional view taken along the line IX-IX in FIG. 8.

FIG. 10 is an entire perspective view showing an exemplary stretcher according to a fourth embodiment.

FIG. 11 is a cross-sectional view taken along the line XI-XI in FIG. 10.

### DETAILED DESCRIPTION OF THE PREFERRED EMBODIMENTS

Various example embodiments will now be described more fully hereinafter with reference to the accompanying drawings, in which exemplary embodiments are shown. The present invention may, however, be embodied in many different forms and should not be construed as limited to the example embodiments set forth herein. That is, these example embodiments are just that—examples—and many implementations and variations are possible that do not require the various details herein. It should also be emphasized that the disclosure provides details of alternative examples, but such listing of alternatives is not exhaustive. Furthermore, any consistency of detail between various examples should not be interpreted as requiring such detail—it is impracticable to list every possible variation for every feature described herein. The language of the claims should be referenced in determining the requirements of the invention. In the drawings, the sizes and relative size of layers and regions may be exaggerated for clarity. Like numerals refer to like elements throughout.

The terminology used herein is for the purpose of describing particular embodiments only and is not intended to limit the invention. As used herein, the singular terms “a,” “an” and “the” should not exclude the plural forms as well, unless the context clearly indicates otherwise. As used herein, the term “and/or” includes any and all combinations of one or more of the associated listed items. It will be understood that when an element or a layer is referred to as being “on,” “connected to” or “coupled to” another element or layer, it may be directly on, connected to, or coupled to the other element or layer or intervening elements or layers may be present. In contrast, when an element is referred to as being “directly on,” “directly connected to” or “directly coupled to” another element or layer, there are no intervening elements or layers present. Other words used to describe the relationship between elements or layers should be interpreted in a like fashion (e.g., “between” versus “directly between;” “adjacent” versus “directly adjacent,” etc.).

It will be further understood that the terms “comprises”, “comprising,” “includes” and/or “including”, when used herein, specify the presence of stated features, integers, steps, operations, elements, and/or components, but do not preclude the presence or addition of one or more other features, integers, steps, operations, elements, components, and/or groups thereof.

Terms such as “same,” “planar,” or “coplanar,” as used herein when referring to orientation, layout, location, shapes, sizes, amounts, or other measures do not necessarily mean an exactly identical orientation, layout, location, shape, size, amount, or other measure, but are intended to encompass nearly identical orientation, layout, location, shapes, sizes, amounts, or other measures within acceptable variations that may occur, for example, due to manufacturing processes.

Example embodiments are described herein with reference to cross-sectional illustrations that are schematic illustrations of idealized example embodiments (and intermediate structures) of example embodiments. As such, variations from the shapes of the illustrations as a result, for example, of manufacturing techniques and/or tolerances, are to be expected. Example embodiments should not be construed as limited to those shown in the views, but include modifications in configuration formed on the basis of, for example, manufacturing processes. For example, a region illustrated as a rectangle may have rounded or curved features. Thus, the regions illustrated in the figures may be schematic in nature and their shapes are not intended to limit the scope of the present disclosure.

It will be also understood that although the terms first, second, third etc. may be used herein to describe various elements, components, regions, layers and/or sections, these elements, components, regions, layers, and/or sections should not be limited by these terms. Unless otherwise indicated, these terms are only used to distinguish one element, component, region, layer, or section from another element, components, region, layer, or section. Thus, a first element, components, region, layer, or section in some embodiments could be termed a second element, components, region, layer, or section in other embodiments, and, similarly, a second element, components, region, layer, or section could be termed a first element, components, region, layer, or section without departing from the teachings of the disclosure. Exemplary embodiments explained and illustrated herein may include their complementary counterparts.

Locational terms, such as “beneath,” “below,” “lower,” “above,” “upper” and the like, may be used herein for ease of description to describe one element or feature's relationship to another element(s) or feature(s) as illustrated in the figures. It will be understood that the locational terms may be relative to a device and are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the locational descriptors used herein interpreted accordingly.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and/or the present application and will not be interpreted in an idealized or overly formal sense unless expressly so defined herein.

Hereinafter, example embodiments will be explained in detail with reference to the accompanying drawings.

Since human beings start bipedal walking, the joints, including, for example, a spine, waist and knee joints, of a human body are compressed by its own weight, which sometimes causes joint problems. As people age, potentially due to the loads applied to a spine or waist or knee joints, for example, of a human body over the years or excessive loads applied thereto, joint problems, such as, e.g., lumbar spinal canal stenosis, spinal disc herniation, osteoarthritis of hip, osteoarthritis, fracture of the femoral neck and medial meniscus injury, may arise.

These joint problems are increasing in number because of, e.g., increasing obesity rate, taking less calcium and diabetes. Once a joint problem occurs, surgery is often considered to be the effective method to cure the problem.

There have been proposed several stretchers configured to stretch a lower body of a human body. In some cases, a stretcher may be designed to stretch a spine, hip joints, and knee joints using the user's own body weight. For example, a stretcher may be configured to hold a user's ankles with the user's head positioned downward. These types of stretchers may be difficult to use on a daily basis due to issues with blood circulation due to the user's posture during use. In some cases, stretchers may be used in a hospital for treating joint problems, e.g., lumbar spinal canal stenosis, or spinal disc herniation. These stretchers may be configured to stretch a spine using a waist belt to which weight members are connected via a cord and pulleys. Often, the stretchers designed for use in a hospital may be large in size and complicated in structure. Using such device can provide a certain level of treatment effects but may not be optimal for daily use, as users may not be able to visit a hospital every day to get treatments.

Joint problems may sometimes be removed immediately after a stretching treatment using a stretcher in a hospital but may return or start again as time passes. For example, reoccurrence of joint problems may be caused by resuming a posture in which loads are applied to joints in a compressing direction immediately after stretching those joints in a stretcher during hospital treatment. An exemplary stretcher may reduce the possibility of occurrence of joint problems or prevent possible reoccurrence of joint problems after a stretching treatment.

### First Embodiment

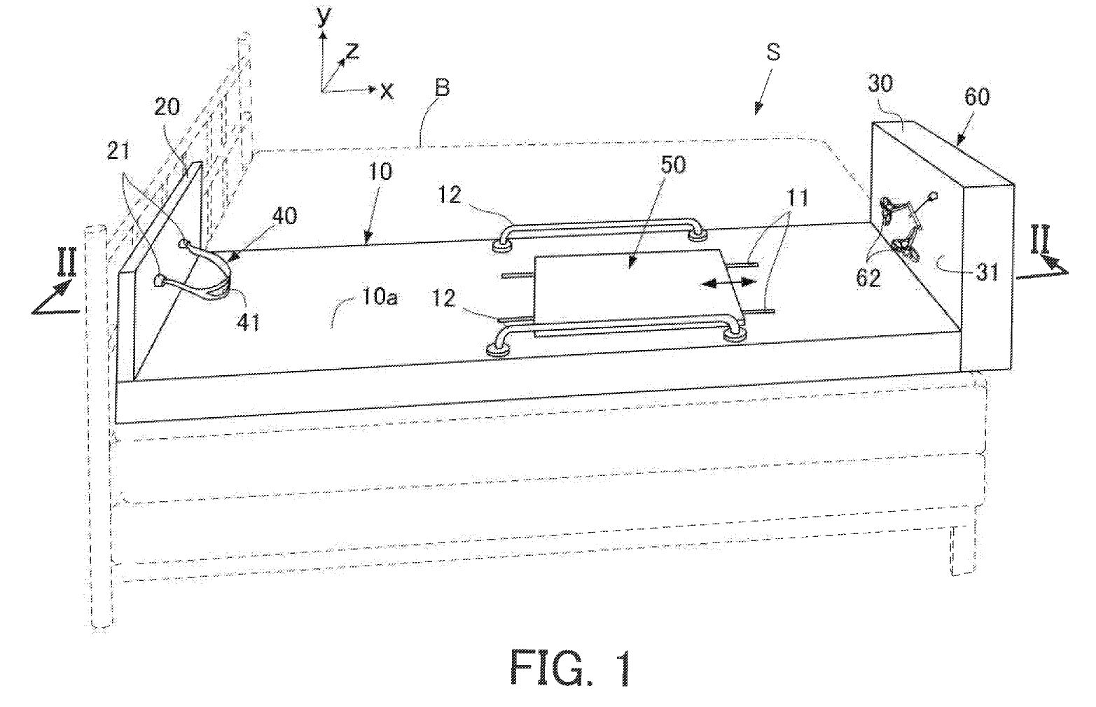

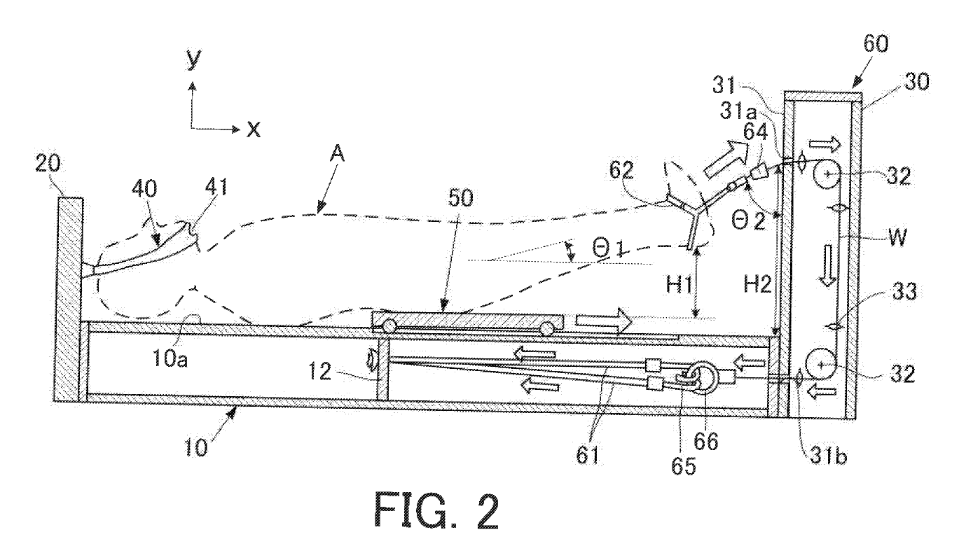

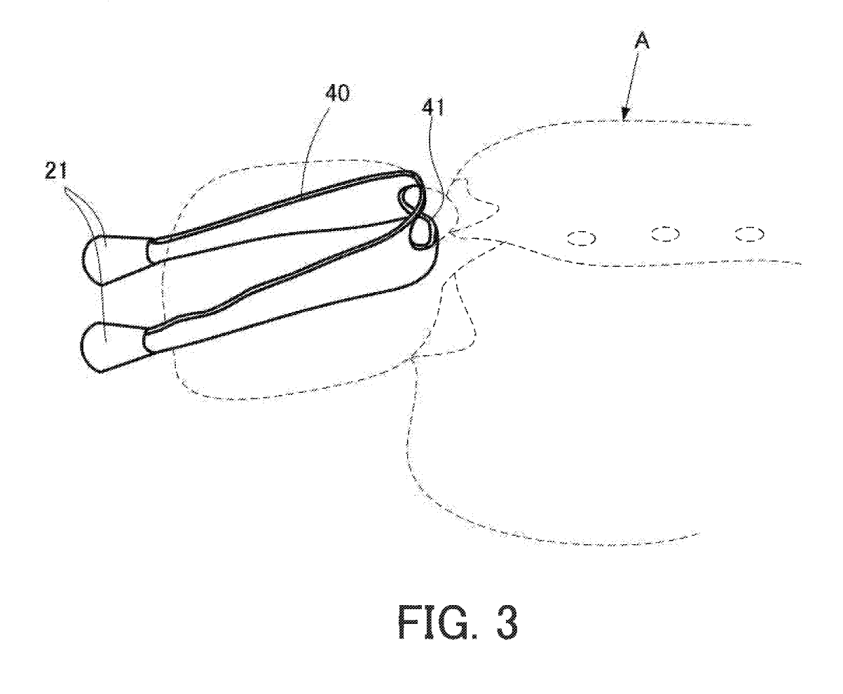

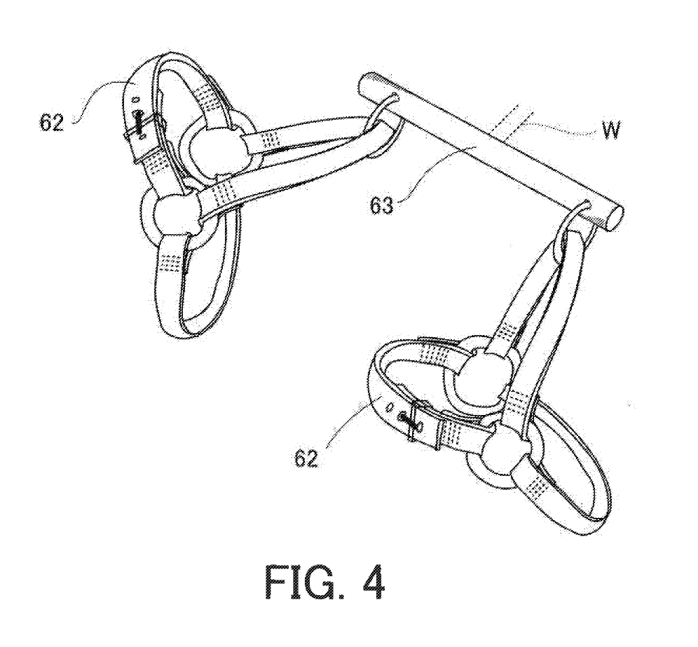

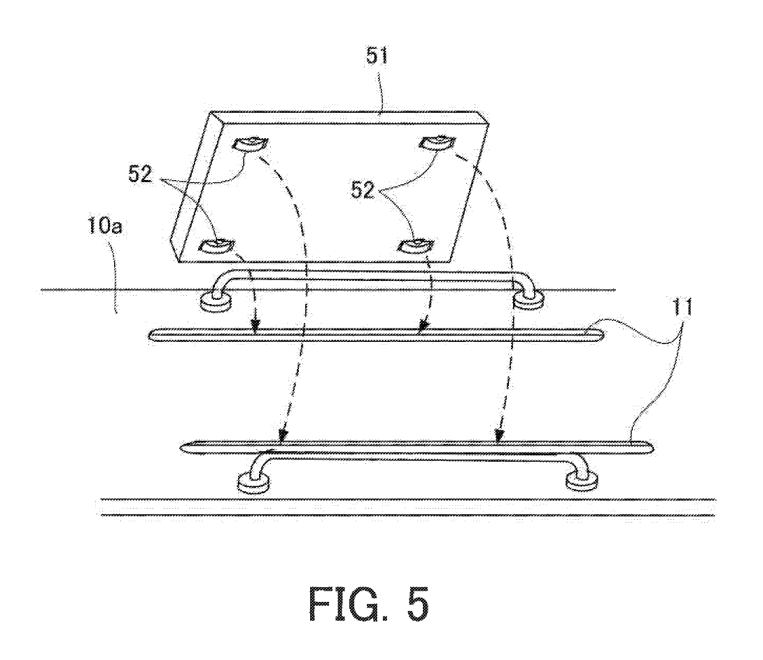

FIGS. 1 to 5 show an exemplary stretcher according to a first embodiment. FIG. 1 is an entire perspective view showing a schematic structure of an exemplary stretcher according to the first embodiment. FIG. 2 is a cross-sectional view taken along the line 2-2 in FIG. 2. FIG. 3 is a perspective view showing an exemplary chin strap as an upper body movement restraint of the stretcher according to the first embodiment. FIG. 4 is a perspective view showing an exemplary ankle strap as a part of a tension generator of the stretcher according to the first embodiment. FIG. 5 is a perspective view showing an exemplary friction reducer of the stretcher according to the first embodiment.

The stretcher S of this embodiment, as shown in FIG. 1, is used, for example, in a state in which the stretcher S is arranged on one side of the upper surface of a bed B. The bed B may be any commercially available bed or custom-made bed. The bed B may be used for sleeping and may have a width (i.e. distance in the z-direction) sufficient for the stretcher S and for a user to lay longitudinally (i.e. in the x-direction) next to the stretcher S. In some embodiments, the bed B may be a bed that includes a mechanism (i.e. a sheet or a flat surface on which a user lays on top of the stretcher) with which a user is capable of being transferred to a second bed without having to stand on the user's feet for transportation. In these embodiments, the bed B may not have a sufficient width to include both the stretcher S and a user laying in longitudinally (in the x-direction). The stretcher S is provided with a base 10, a head board 20 attached to one end (i.e., the head side) of the base 10, a foot board 30 (including, for example, a cord guide portion) attached to the other end (i.e., the foot side) of the base 10, an upper body movement restraint 40, a friction reducer 50, and a tension generator 60.

The base 10 may be formed into a rectangular shape having a sufficient length and a width for a user (e.g. a fully grown human adult) to lay down thereon. In some embodiments, the base 10 is placed on the bed B. As shown in FIGS. 1 and 2, within the inner space of the base 10, at least a portion of a tension generator 60, which will be explained later, is accommodated.

In some embodiments, the head board 20 is fixed to one end (i.e., the head side end) of the base 10 and extends upwardly in a vertical direction (i.e., the y-direction). The head board 20 may be provided with a pair of right and left fixants 21 at a front surface of the head board 20, the front surface facing the foot board 30 of the stretcher S. In other embodiments, the fixants 21 may be affixed to a left and right side of the base 10. The fixants 21 attach both ends of a chin strap 40 (used as an upper body movement restraint) to the head board 20. The fixants 21 may be, for example, handles, loopholes, or other connectors affixed to the head board 20 that connect the upper body movement restraint 40 to the head board 20. The fixants 21 may include an adjustment mechanism, such that the chin strap 40 may be shortened or lengthened to be adjusted to the chin and head of a user to snugly fit a user's head and chin without causing pain. In some embodiments, the chin strap 40 itself may be adjusted to the chin and head of a user to snugly fit a user's head and chin without causing pain. The upper body movement restraint 40 may restrain the head and/or upper body of a user from moving in an x-direction away from the head board 20. In some embodiments, the upper body movement restraint 40 may not restrain the head and/or upper body of the user from moving in an x-direction towards the head board 20. In some embodiments, the fixants 21 may be attached to a section of the base 10 adjacent the head side of the base.

As shown in FIG. 3, the chin strap 40 is a single piece of material with one end fixedly connected to the right fixant 21 of the head board 20 and the other end fixedly connected to the left fixant 21 of the head board 20. In some embodiments, the chin strap 40 may be shaped like a belt. In some embodiments, the chin strap may be a single piece of material with a consistent depth, but which increases in width towards the middle of the strap. In these embodiments, the chin strap 40 is narrower at the ends and wider at the middle of the strap 40. As mentioned above, the length of the chin strap 40 may be adjustable. For example, the chin strap 40 may include adjustment members that are used to adjust the length of the chin strap 40 for a particular user. In another example, the fixants 21 may be used to adjust the length of the chin strap 40.

The chin strap 40 may be made of cloth, leather, plastic, or any other suitable material that may be used to contact human skin. In some embodiments, the chin strap 40 may include a softer material on the inside of the chin strap 40 which contacts the user's skin, and may have a harder, less flexible material on the outside of the chin strap 40 to ensure that the user's head is not able to move significantly. For example, the outer material of the chin strap 40 may be less flexible, such that the user's head, when in the chin strap 40, may not move more than 1 inch in an z-direction or move in an x-direction such that the top of the user's head is more than 1 inch from a closest surface of the head board 20. In some examples, the user's head may not be able to move more than ½ inch or ¼ inch in a z-direction or move in an x-direction such that the top of the user's head is more than 1 inch from a closest surface of the head board 20 when disposed in the chin strap 40. The chin strap 40 may restrict the movement of the user's head such that it may not be move in the x-direction past a predetermined area of the base 10. The chin strap 40 may include an opening 41 in which to fit a chin (or jaw) of a user. The opening 41 may be disposed in the middle of the chin strap and may be formed along the length of the chip strap. In some embodiments, the chin strap 40 does not include an opening 41.

In some embodiments, the upper body movement restraint 40 is a chin strap as shown in FIG. 3. In other embodiments, other straps, belts, rigid restraints or other mechanisms may be used as the upper body restraint 40. The structure, shape, and material of the upper body restraint 40 is not limited to the exemplified chin strap, but may be limited to those combinations of structures, shapes, and materials configured to prevent the upper body of the user lying down on the base 10 from being moved in a stretching direction. In another example, the upper body restraint 40 can be a pair of right and left cords or ropes provided on the upper surface of the widthwise sides of the base 10 in an upwardly protruded manner so as to hold the user's under arm portions. Similar cords or other restraints may be used across a user's chest, along other areas of the user's arms, and so forth.

As shown in FIG. 1, a friction reducer 50 is disposed on a portion of an upper surface 10a of the base 10 on which a part of the lower body (e.g., the waist portion or hip portion) of the user is to be laid. The friction reducer 50 may be a slide plate 50 that is movable in a longitudinal direction (e.g. the x-direction). As shown in FIG. 5, in one embodiment, the slide plate 50 is provided with rollers 52 disposed at the four corner portions of the lower surface of the body 51 of the slide plate 50. Corresponding to these rollers 52, a pair of guide grooves 11 are formed on the upper surface 10a of the base 10 so as to extend in the x-direction and parallel to each other at the base 10. The body 51 of the plate 50 is disposed on the base 10 with the rollers 52 fitted in the guide grooves 11.

The slide plate 50 may be disposed on the base 10 in a slidable manner. For example, the slide plate may slide from a first end of the guide grooves 11 to the other end of the guide grooves 11. In some embodiments, the slide plate 50 may encounter less friction along the guide grooves 11 than the friction between a user body disposed directly on a portion of the upper surface 10a. In these embodiments, the stretching operation using this stretcher S is enhanced. The upper surface of the slide plate 50 may be covered with a surface material with a high friction coefficient. The surface material of the slide plate 50 may also be a material that is thick and/or soft, so as to be palatable to a user's touch. In some embodiments, a filler material may be placed between the surface material and the upper surface of the slide plate 50 to provide a cushion to the user.

In some embodiments, the friction reducer 50 may be a slide plate (such as the slide plate 50 described above). In other embodiments, the friction reducer 50 may be a plate having a small friction coefficient and having no rollers. Alternatively, the upper surface 10a of the base 10 may include a first section disposed at a part of the upper surface at which a user's upper body would be disposed and a second section disposed at a part of the upper surface at which a user's lower boy would be disposed. In some embodiments, the first section of the upper surface 10a may have a friction coefficient F1 and the second section of the upper surface 10a may have a friction coefficient F2, where F1 is greater than F2. For example, the first section may have a friction coefficient that is at least 5 times larger than the friction coefficient of the second section. In other examples, the first section may have a friction coefficient that is at least twice as large as the friction coefficient of the second section. In some embodiments, the ratio of F1/F2 may range between 1.5 and 10. In some embodiments, the upper body of the user may be prevented from being moved in the x-direction due to the variance in friction coefficient from the first section to the second section. In some of these embodiments, the lower body (or a portion thereof) may move in the x-direction while the upper body is prevented from moving during the stretching operation.

As shown in FIG. 1, the base 10 is provided, on its upper surface 10a, with a pair of right and left grip members 12 arranged at the outer sides of the guide grooves 11. The user can correct his/her laying position by holding the grip members 12.

A first end of foot board 30 (including a cord guide) is fixed to the other end portion (foot side) of the base 10. In some embodiments, only a cord guide portion is used. In other embodiments, the cord guide is an integral part of the foot board 30. The cord guide may be a foot board, a pole, a support member, a beam, or any other suitable device that includes a front surface facing the head side of the base 10, with an opening disposed thereon for a cord, and sufficient space to include therein a cord and one or more pulleys. The size, shape, and dimensions of the cord guide are not limited to the examples described herein. The cord guide may extend upward adjacent to the foot end of the base 10, but may not be perpendicular to the upper surface 10a of the base. In some embodiments, the cord guide may be perpendicular to the upper surface 10a of the base. In some embodiments, the cord guide may be curved. In other embodiments, the cord guide may be straight.

In one embodiment, the first end of the foot board 30 is attached to a foot side of the base 10 such that the entire outer surface of the foot side end of the base 10 contacts a lower portion of the front wall 31 of the foot board 30. In some embodiments, only an upper edge of the outer surface of the second end (e.g., foot side end) of the base 10 contacts a lower edge of the front wall 31. The foot board 30 may protrude upwardly in a vertical direction (e.g. the y-direction) and may be perpendicular to the upper surface 10a of the base 10.

The foot board 30 may be provided with an upper cord insertion hole 31a and a lower cord insertion hole 31b. The upper cord insertion hole 31a may be disposed in the middle of the front wall 31 of the foot board 30 in the z-direction and in at least the upper half of the front wall 31 with regard to the height of the foot board 30 (e.g. in the y-direction) and may be any opening suitable for a cord to pass therethrough. In some embodiments, the upper cord insertion hole 31a may be disposed in the upper quarter of the front wall 31 with regard to an overall height of the front wall, or with regard to a height of the front wall above the top of the base 10 and may be any opening suitable for a cord to pass therethrough. Within the foot board 30, a pair of upper and lower pulleys 32 are disposed. The front wall 31 of the foot board 30, which faces toward the head board 20, has, at its lower end portion, a lower cord insertion hole 31b. The lower cord insertion hole 31b may be at a lower portion of the front wall 31 that is in contact with an outer surface of the base 10. In some embodiments, the lower cord insertion hole 31b may be disposed in the middle of the front wall 31 of the foot board 30 with regard to width (e.g. in the z-direction) and in the middle of the portion of the front wall 31 in contact with the base 10 with regard to the height of that portion.

As shown in FIG. 2, one end of the cord is attached to right and left ankle straps 62. The ankle straps 62 may also be foot straps or any other straps or cords that securely wrap around a portion of the user's lower body. For example, they may wrap around a user's foot, a user's ankle, a user's shin, a user's knees, etc. In some embodiments, only one strap may be used. The cord may be a wire W, as shown in FIG. 2. In other embodiments, the cord may be a cable, a rope, a chain, strands of wire meshed together, string, or other thin, elongated and bendable material for connecting the ankle straps 62 to a tension generating member 61. Specifically, as shown in FIG. 4, the right and left ankle straps 62 are connected to a right and left end respectively of a short bar 63, to which one end of the cord is also connected. At one end of the cord, a pair of right and left ankle straps 62 are attached.

As shown in FIG. 4, for example, the left and right ankle straps 62 are designed to detachably hold, for example, the user's ankle. The ankle straps 62 may include a material such as a rope, a belt, a cloth, a leash-like material, etc. The outer surface of the ankle straps 62 that would be in contact with the user's body may include a softer material, such that the ankle strap 62 does not cut or otherwise rub against or burn the user's foot or leg when attached to the user. For example, the softer material may include a gel, a cloth, padding, or other suitable material that would not chafe against or in any way injure a user's skin. The ankle straps may be adjustable in length so as to best fit a user's ankles. In some embodiments, a stopper 64 is disposed near the end of the cord connected to the short bar 63. The stopper 64 has a diameter larger than the diameter of the upper cord insertion hole 31a. In an embodiment in which the stretcher S is not in use, the stopper 64 is engaged with the front wall 31 of the foot board 30 to prevent the cord from being further pulled into the foot board 30.

In some embodiments, the ankle straps 62 are designed to have an easy release mechanism. In some embodiments, the user does not have to leave a laying posture to disengage from the ankle straps. For example, the ankle straps may be removed by a toe release method. The toe release method may involve a user using a toe (i.e. the big toe) of one foot to remove an ankle strap on the other foot. In other embodiments, the ankle straps may be of a similar shape and dimension to a sock. In these embodiments, the ankle straps may again be removed by a toe release method or other suitable easy release method. In some embodiments, when the user's legs are disposed in the ankle straps 62, and the user's legs are performing a pull motion, the straps may cinch around the user's foot. When the user's legs are disposed in the ankle straps 62, and the user's legs are not performing a pull motion, or when the user's legs perform a push motion, the ankle straps may open up and may easily release the user's foot. For example, when the user's legs perform a push motion, the ankle straps may be disposed such that the user's foot may fall out of the ankle strap through an opening. In other examples, the ankle straps may create an opening through which the user's foot could easily slide out of the ankle straps. In some embodiments, the ankle strap may only open up and easily release the user's foot when the user's legs perform a push motion.

In some embodiments, the cord extends into the inner space of the foot board 30 via the upper cord insertion hole 31a, engages with upper and lower pulleys 32 and then extends into the inner space of the base 10 via the lower cord insertion hole 31b. In the base 10, two rubber cords 61 act as tension generating members. More than two rubber cords 61, or only one rubber cord 61, may be used as tension generating member(s). The two rubber cords 61 may be disposed with one end connected to an intermediate wall 12 of the base 10 and the other end having a hook member 65 detachably engaged with a ring member 66 connected to the other end of the cord.

The intermediate wall 12 of the base may extend from an upper inner surface of the base 10 to a lower inner surface of the base in the y-direction. In some embodiments, the intermediate wall 12 of the base may be disposed under an end of the slide plate 50 closest to the head board 20. In other embodiments, the intermediate wall 12 of the base may be disposed in the base 10 such that the two rubber cords 61 are stretched to at least 75% of their maximum elasticity when the stretcher S is not in use. In some embodiments, by changing the number of the rubber cords 61 in the base 10, the tension applied to the cord can be changed, thereby adjusting the stretching force of the tension generator 60. In some embodiments, the tension generating member 61 is a rubber cord such as rubber cord 61 described above. In other embodiments, the tension generating member 61 is, for example, a coil spring or a device configured to pull the cord using an electric motor or a hydraulic piston. In some embodiments, the tension generator 60 comprises the left and right ankle straps 62, the cord, the upper and lower pulleys 32, and the tension generating member(s) 61.

An exemplary method of operating the stretcher S of this embodiment follows. As shown in FIG. 1, the stretcher S is arranged on one side of the bed B. As shown in FIG. 2, a user A lays down on the stretcher S such that a portion of the upper body of the user A is held with his/her chin hooked by the chin strap 40 and the ankles of the user A are held by the left and right ankle straps 62 with the waist or hip portion of the user A arranged on the slide plate 50. With the user A attached to the stretcher S in this manner, the tension of the cord is increased. In some embodiments, the rubber cords 61 and the ankle straps 62 are pulled with the cord and the ankles of the user A, which are held by the left and right ankle straps 62, are pulled obliquely upward. In this position, since the waist or hip portion of the user A is arranged on the slide plate 50, the lower body of the user A will be pulled toward the foot board 30 together with the slide plate 50. This results in effective stretching of the lower body of the user A.

In some embodiments, and as shown in FIG. 2, the tension generator 60 is configured to stretch the lower body of the user such that the user's lower body (e.g., the user's legs) ascends at a angle θ1 with respect to the upper surface 10a of the base 10. The angle θ1 may range, for example, from 5 to 45 degrees. In some embodiments, the angle θ1 is an angle similar to the angle of a user's legs when the user takes a step. This embodiment is particularly useful because these angles may provide for optimal stretching in a natural body position. For example, the angle θ1 is 10 degrees, and may be within a range of 8 to 15 degrees. With this ascending angle θ1, joints of the lower body of the user A including the spine, hip joints, knee joints, etc., can be effectively stretched.

In some embodiments, the height H1 of the heel of the user A above the upper surface 10a of the base 10 ranges from 5 to 50 cm. In some embodiments, the height H1 may be the distance between a user's feet (e.g. a front of a back foot and a back of the foot in front of it) when the user takes a step. These embodiments may be particularly useful, as they may provide a angle θ1 that is optimal for stretching. For example, the height H1 of the heel of the user A above the upper surface 10a of the base 10 may be at least 10 cm (centimeters) and may range from 5 to 25 cm.

In some embodiments, the height H2 of the cord insertion hole 31a may range from 15 to 75 cm. In some embodiments, the height H2 may be 25 cm above the upper surface 10a of the base 10, and may range from 20 to 45 cm. These embodiments may be particularly useful because the ascending angle θ1 of the user's leg with respect to the base 10 may depend upon the height H2 of the cord insertion hole 31a. The height H2 may be adjusted based on the height of the user. In some embodiments, the height H2 may be fixed and may not be adjustable.

In some embodiments, the angle θ1 may be 0 (zero). In these embodiments, the lower body is pulled horizontally instead of at a angle.

In some embodiments, in order to facilitate the attaching operation of the foot strap 62 to the ankle portion of the user A, the cord insertion hole 31a is configured such that a friction between the cord insertion hole 31a and the cord when not in use is set to be larger than a friction between the cord insertion hole 31a and the cord during the stretching operation. In these embodiments, the depression angle θ2 of the cord may be smaller during stretching (e.g. during use by the user A) than when not in use. When a user's ankles are attached to the ankle straps 62, the cord may be pulled out from the foot board 30 and may be bent downward so that the depression angle θ2 of the cord becomes smaller. The friction between the cord insertion hole 31a and the cord is large, and therefore the user can readily attach the ankle strap 62. After attaching the ankle strap 62, the lower leg portion with the ankle strap 62 is raised to increase the depression angle θ2, resulting in a reduced friction between the cord insertion hole 31a and the cord. This in turn allows the cord to pull the lower leg portion of the user with full strength.

The stretching operation using this stretcher S is to be performed for a certain period of time. For example, the user may use the stretcher S for at least 15 to 30 minutes. The user may also sleep through the night while in the stretcher S or may remain in the stretcher for a longer period of time. In some embodiments, the stretching operation may include the user laying or sleeping in the stretcher. In some embodiments, the stretching operation may also include the user exercising in the stretcher S for a certain period of time. For example, the user may move his legs back and forth in the x-direction while attached to the stretcher to exercise while using the stretcher S. In other examples, the user may move his lower body in the x-direction on the slidable plate or other friction reducer to exercise while using the stretcher S.

After completion of the stretching operation, the chin strap 40 and the ankle strap 62 are detached from the user A, and the user A may move to the other side of the bed B (i.e. by rolling over), maintaining a laying posture for at least a certain period of time or for an entire night of sleep on the bed B. In some embodiments, the user A may sleep in the stretcher S and may move to another side of the bed B and may maintain a laying posture for a certain amount of time. For example, the user may maintain a laying posture for at least 15 minutes after the stretching operation. In some embodiments, the user may maintain a laying posture for at least 30 minutes after the stretching operation. This effectively prevents joints of the user A from being pressed in the compressing direction even after the stretching operation, resulting in remarkable benefits for treatment. In some embodiments, the stretcher S is arranged on the bed B or arranged adjacent to the bed B with the upper surface of the stretcher S flush with the upper surface of the bed B so that the user A can immediately move on the bed after completion of the stretching operation using the stretcher S.

### Second Embodiment

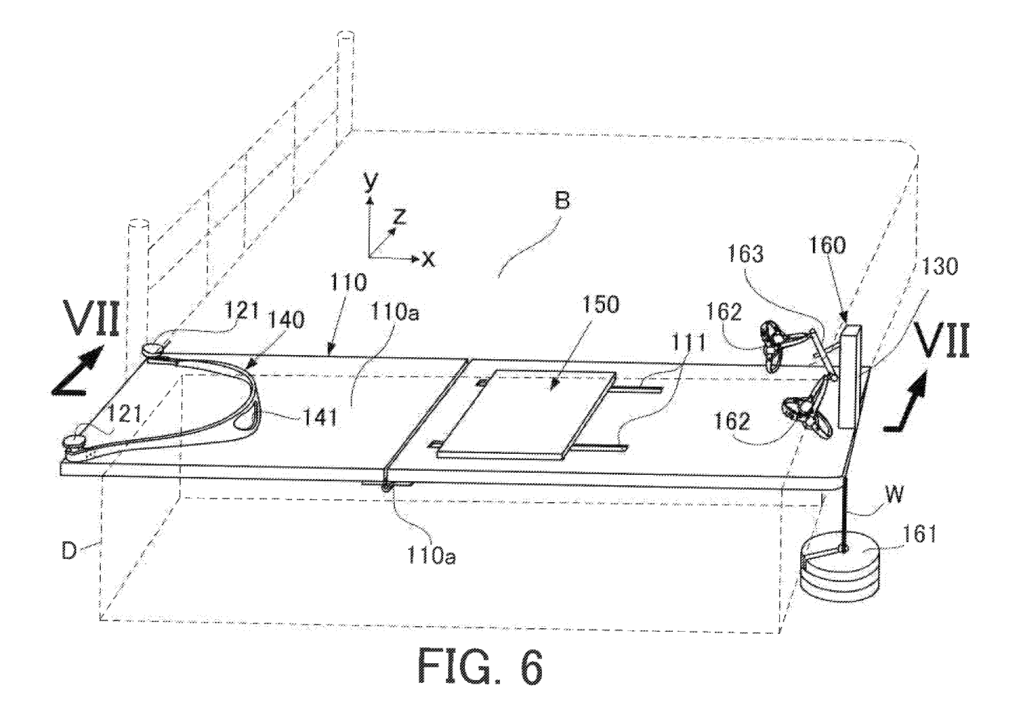

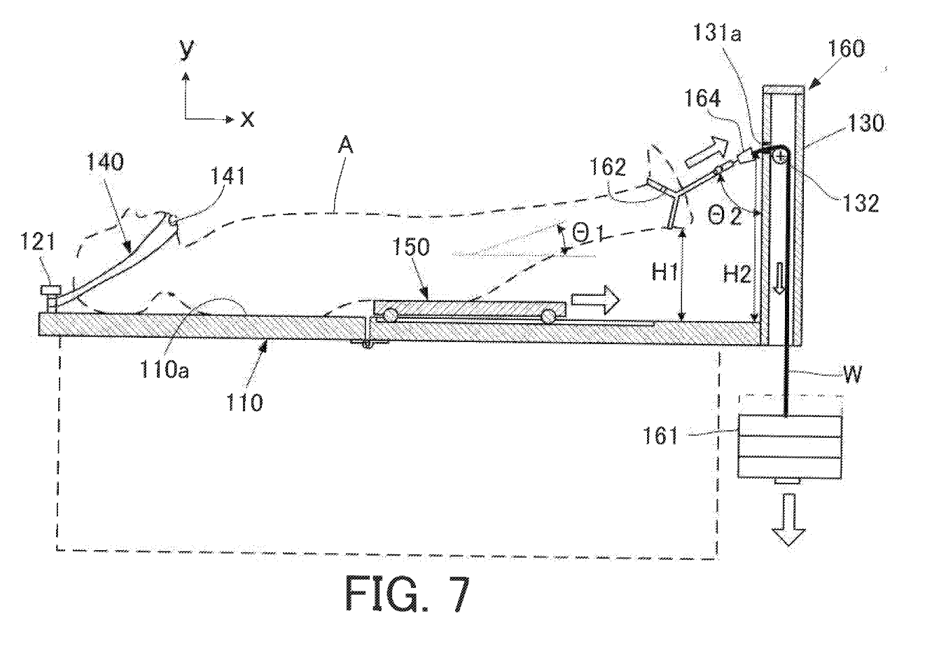

FIGS. 6 and 7 show a second embodiment. FIG. 6 is an entire perspective view showing a stretcher according to the second embodiment, and FIG. 7 is a cross-sectional view taken along the line VII-VII in FIG. 6.

The stretcher S of this embodiment, as shown in FIG. 6, is used in such a manner that the stretcher S is arranged on a table D arranged adjacent to the bed B with the upper surface of the stretcher S being approximately flush with the upper surface of the bed B. In some embodiments, the stretcher S includes a base 110, a pair of right and left hooks 121 attached to a first end of the base 110, an upper body movement restraint 140 having both ends attached to the hooks 121, a pole 130 as a cord guide attached to a second end of the base 110 opposite the first end, a slide plate 150 as a friction reducer disposed on an upper surface on the based 110, left and right ankle straps 162, and a tension generator 160 including a cord and weight members 161.

In some embodiments, the base 110 includes a first portion and a second portion, where the first portion is connected via a hinge member 110a to the second portion. The stretcher S may fold at the hinge member 110. In some embodiments, when not in use, the stretcher S can be stored in a compact folded manner.

The upper body movement restraint 140, the friction reducer 150, and the left and right ankle straps 162 are similar to the upper body movement restraint 40, the friction reducer 50, and the left and right ankle straps 62, respectively.

In some embodiments, the cord is stretched (and stretches the left and right ankle straps 162) using the weight of the weight members 161. A pulley 132 is disposed in the pole 130. In some embodiments, the cord extends into the pole 130 via a cord insertion hole 131a formed in the front wall of the pole 130 and extends to the weight member 161 via one or more pulleys 132.

An exemplary method of operating the stretcher S of this embodiment follows. The user may lay in the stretcher, attached to the upper body movement restraint 140 and left and right ankle straps 162 and disposed with a portion of the user's lower body on the friction reducer 150 in a manner the same or similar to the first embodiments. In some embodiments, once attached to the stretcher, the cord is pulled downward by the weight members 161, and the lower leg portions of the user A, which are held by the left and right ankle straps 62, are pulled obliquely upward. In this position, since the waist or hip portion of the user A is arranged on the slide plate 50, the lower body of the user A will be pulled toward the pole 130 together with the slide plate 50. As a result, the lower body of the user A is effectively stretched.

The heights H1, H2 and the angles θ1 and θ2 may be the same or similar to the same heights and angles of the first embodiments. Other dimensions discussed with respect to the first embodiment may be the same or similar to respective dimensions of the second embodiment.

The stretching operation using this stretcher S is to be performed for a certain period of time. For example, the user may use the stretcher S for at least 15 to 30 minutes. The user may also sleep through the night while in the stretcher S or may remain in the stretcher for a longer period of time. In some embodiments, the stretching operation may include the user laying or sleeping in the stretcher. In some embodiments, the stretching operation may also include the user exercising in the stretcher S for a certain period of time. For example, the user may move his legs back and forth in the x-direction while attached to the stretcher to exercise while using the stretcher S. In other examples, the user may move his lower body in the x-direction on the slidable plate or other friction reducer to exercise while using the stretcher S.

After completion of the stretching operation, the chin strap 40 and the ankle strap 62 are detached from the user A, and the user A may move to the other side of the bed B (i.e. by rolling over), maintaining a laying posture for at least a certain period of time or for an entire night of sleep on the bed B. In some embodiments, the user A may sleep in the stretcher S and may move to another side of the bed B and may maintain a laying posture for a certain amount of time. For example, the user may maintain a laying posture for at least 15 minutes after the stretching operation. In some embodiments, the user may maintain a laying posture for at least 30 minutes after the stretching operation. This effectively prevents joints of the user A from being pressed in the compressing direction even after the stretching operation, resulting in remarkable benefits for treatment. In some embodiments, the stretcher S is arranged on the bed B or arranged adjacent to the bed B with the upper surface of the stretcher S flush with the upper surface of the bed B so that the user A can immediately move on the bed after completion of the stretching operation using the stretcher S.

### Third Embodiment

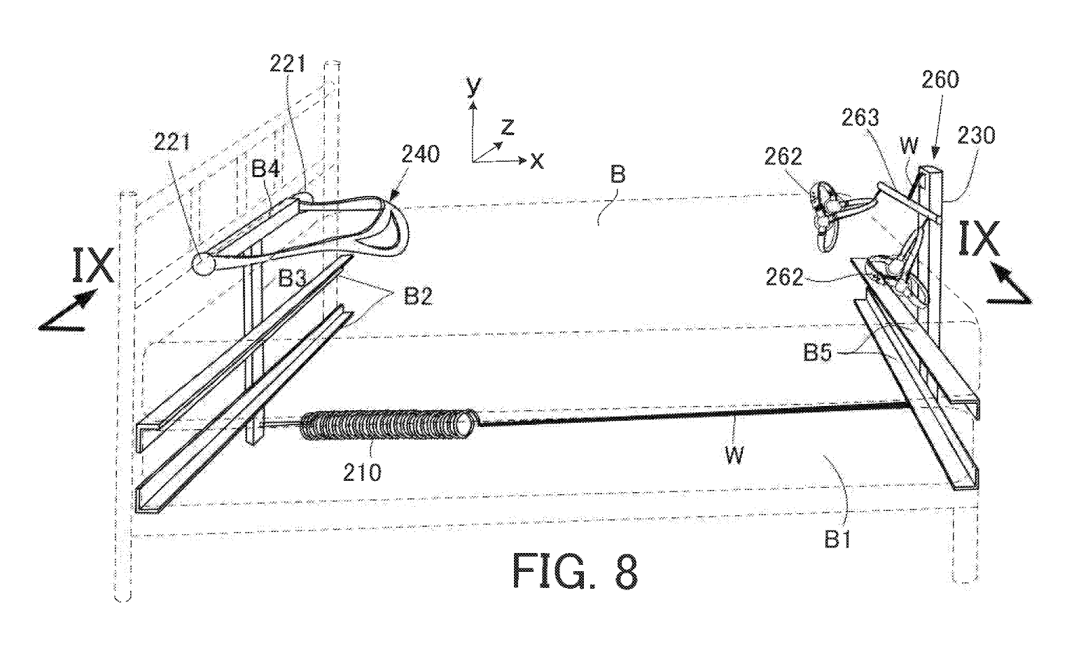

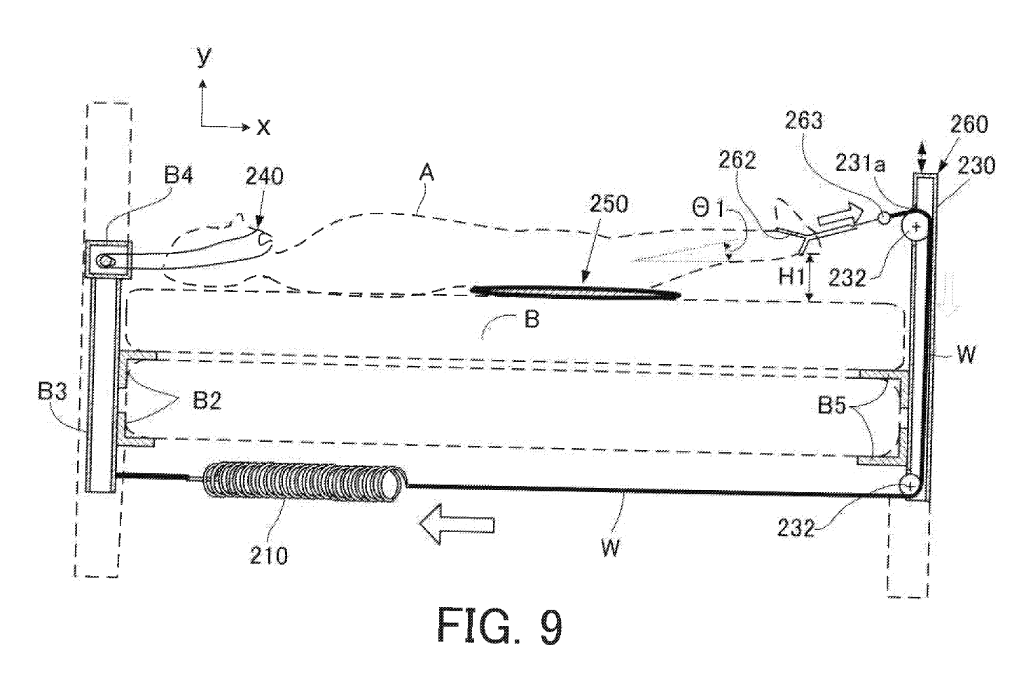

FIGS. 8 and 9 show a stretcher S according to a third embodiment. FIG. 8 is an perspective view showing a stretcher according to this embodiment. FIG. 9 is a cross-sectional view taken along the line IX-IX in FIG. 8.

This stretcher S of this embodiment is integrally mounted on one side of the bed B. In some embodiments, the stretcher S includes an upper body movement restraint 240, a tension generator 260, and a friction reducer 250.

In the stretcher S of this embodiment, a pair of upper and lower angled ends B2 are fixed to upper and lower potions of a first end (head side) of a base B1 of the bed B. The upper and lower angled ends B2 and the upper and lower angled ends B4 (discussed below) may comprise a box spring of a bed, upon which a mattress may be placed. The upper angled end B2 may be disposed at an upper outside corner of the base B1. The lower angled end B2 may be disposed at a lower outside corner of the base B1. In some embodiments, the outer surface of upper outside corner the base B1 may be covered by the upper angled end B2. In some embodiments, the outer surface of the lower outside corner of the base B1 may be covered by the lower angled end B2. In some embodiments, the angled ends B2 may extend in the z-direction, such that the depth of the angled ends B2 is the same as the depth of the base B1 and/or the bed B.

A vertical member B3 is fixed to the angled ends B2. The vertical member B3 may be fixed at a center in the z-direction of the angled ends B2. One end of the vertical member B3 may extend to the ground or may extend downward for a same amount as the bed B. The other end of the vertical member B3 may extend upward such that the end is above the upper surface of the bed B.

A horizontal member B4 is fixed to a top portion of the vertical member B3, such that the center (in the z-direction) of the horizontal member B4 is fixed to the top portion of the vertical member B3. The horizontal member B4 may be parallel in the z-direction with the angled ends B2, but may not extend in the z-direction like the angled ends B2. The width in the z-direction of the horizontal member B4 may be less than ½ or less than ⅓ of that of the angled ends B2. The width in the z-direction of the horizontal member B4 may be at least the size of an adult human head, measured from ear to ear, or at least twice that size. The horizontal member B4 has, at each of its end portions, a pair of hooks 221 to which the ends of an upper body movement restraint 240 (e.g. a chin strap) are hooked. The upper body movement restraint 240 is the same or similar to the upper body movement restraint 40.

Another pair of upper and lower angled ends B5 are fixed to the second end (the foot side) of the bed base B1. The angled ends B5 may be the same or similar to the angled ends B2, except disposed in an opposite direction facing the angled ends B1. A rectangular pipe 230 is fixed to the angled ends B5 and extends upward in a vertical direction (e.g. the y-direction). The rectangular pipe 230 may be disposed at a center in the z-direction of the angled ends B5. In the same or similar manner as in the second embodiment, a cord is arranged in the rectangular pipe member 230 as a cord guide portion. One end of the cord is connected to a pair of ankle straps 262 and the other end of the cord is connected to one end of a tension generator 210, for example, a coil spring or rubber cord disposed at the bottom member B1 of the bed B. In this embodiment, the cord guide is integral to the foot end of the bed.

In the stretcher S of this embodiment, in the same manner as in the first embodiment, as shown in FIG. 9, the user A lays down on the bed B in such a manner that the chin of the user A is hooked with the chin strap 240 and the ankle portions of the user A are held by the ankle straps 262, with the user's waist or hip portion arranged on a friction reducer 250 (e.g. a small mat, a slidable plate, etc.) with a small friction coefficient. In an embodiment in which the user is disposed in the stretcher S, the cord is pulled downward by the coil spring 210, resulting in an obliquely upward pulling of the ankle straps 262. As a result, the lower leg portions of the user A held by the ankle straps 262 are pulled obliquely upward. In this embodiment, since the lower body portion of the user A, i.e., the waist or hip portion, is arranged on the friction reducer 250, the lower body of the user A will be pulled in a direction towards the pole 130. As a result, the lower body of the user A is effectively stretched.

The stretching operation using this stretcher S is to be performed for a certain period of time. For example, the user may use the stretcher S for at least 15 to 30 minutes. The user may also sleep through the night while in the stretcher S or may remain in the stretcher for a longer period of time. In some embodiments, the stretching operation may include the user laying or sleeping in the stretcher. In some embodiments, the stretching operation may also include the user exercising in the stretcher S for a certain period of time. For example, the user may move his legs back and forth in the x-direction while attached to the stretcher to exercise while using the stretcher S. In other examples, the user may move his lower body in the x-direction on the slidable plate or other friction reducer to exercise while using the stretcher S.

After completion of the stretching operation, the chin strap 40 and the ankle strap 62 are detached from the user A, and the user A may move to the other side of the bed B (i.e. by rolling over), maintaining a laying posture for at least a certain period of time or for an entire night of sleep on the bed B. In some embodiments, the user A may sleep in the stretcher S and may move to another side of the bed B and may maintain a laying posture for a certain amount of time. For example, the user may maintain a laying posture for at least 15 minutes after the stretching operation. In some embodiments, the user may maintain a laying posture for at least 30 minutes after the stretching operation. This effectively prevents joints of the user A from being pressed in the compressing direction even after the stretching operation, resulting in remarkable benefits for treatment. In these embodiments, the user is not required to immediately move on a bed after completion of the stretching operation.

The heights H1, H2 and the angles θ1 and θ2 may be the same or similar to the same heights and angles of the first embodiments. Other dimensions discussed with respect to the first embodiment may be the same or similar to respective dimensions of the third embodiment.

The other structures are the same as those of the first embodiment, and therefore the explanation will be omitted by allotting the same or corresponding reference numeral to the corresponding portion.

### Fourth Embodiment

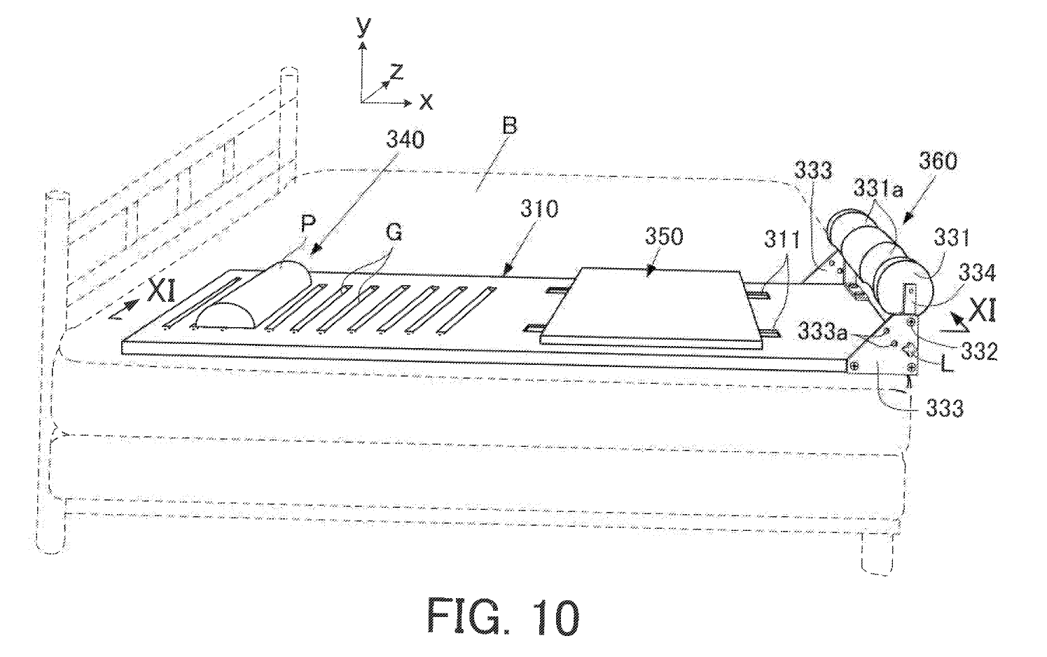

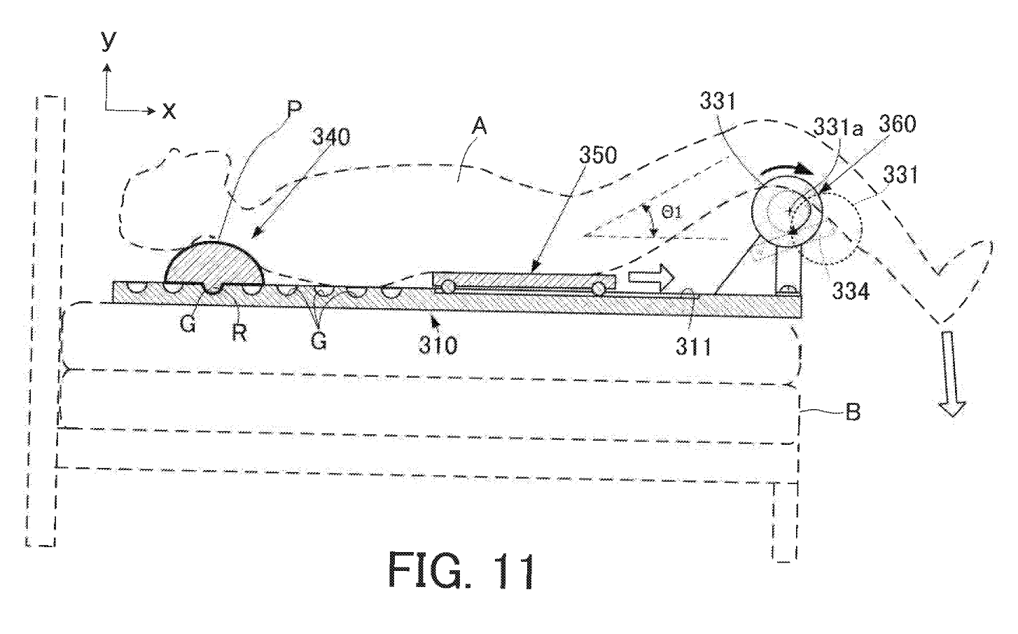

FIGS. 10 and 11 show a stretcher S according to a fourth embodiment. FIG. 10 is an entire perspective view showing the stretcher according to this embodiment, and FIG. 11 is a cross-sectional view taken along the line XI-XI in FIG. 10.

This stretcher S of this embodiment is provided with a base 310, a pillow member P, a friction reducer 350 (e.g. a slide plate or mat), and a tension generator 360 (e.g. a roller).

In this embodiment, an upper body restraint may be the pillow member P arranged on the base 310 at a first end (the head side) of the base 310. The position of the pillow member P may be changed, as shown in FIG. 10, by the plurality of grooves G into which the pillow member may be disposed. The plurality of grooves G may be disposed at a first section of the base 310, at which the upper body of the user may be disposed. The grooves G may be evenly spaced apart protrusions extending upwardly out of the base 10. In some embodiments, the grooves G may extend upwardly from the base at a top height ranging from 0.1 to 3 cms. The grooves G may also be evenly spaced apart indentations into the base, such that the upper surface 10a of the base 10 is at a height ranging from 1 to 3 cms higher than a deepest part of a groove (i.e. the lowest part of the groove in the y-direction).

The plurality of grooves G may be disposed at a center in the x-direction of the first section of the base 310, across half of the surface area of the first section. In some embodiments, the grooves G may be disposed across 75% of the surface area of the first section. Once the pillow member is disposed in a groove, the pillow member P may not be movable unless removed and disposed in another groove when not using the stretcher S. The pillow member P may be formed into a semicircular shape in cross-section and has, at its lower surface, a ridge portion R extending in a z-direction of the pillow member P. The shape of the pillow member P is not limited to the examples described herein. The base 310 has, at its upper surface, a plurality of grooves G, with each groove corresponding to a ridge portion R that is arranged at certain intervals on the lower surface of the pillow member P. By fitting the ridge portions R of the pillow member P to one or more of the grooves G, the pillow member P will be prevented from being moved during the stretching operation. By selecting the fitting position of the ridge R to the groove G, the position of the head can be adjusted depending on the user.

With respect to the friction reducer 350, since the structure is the same as that of the first embodiment, the explanation will be omitted. As mentioned above, the friction reducer 350 is disposed in a second section of the base 310, at which the lower body of the user may be disposed.

The tension generator 360 (e.g. a roller) includes a cylindrical roller main body 331, a pair of right and left mounts 333, and a pair of right and left supporting arms 334. Each supporting arm 334 is rotatably attached to a respective mount 333 by a bolt 332 at the center of the mount 333 in the y-direction. The upper end of the supporting arm 334 is rotatably attached to the roller main body 331. The mount 333 is provided with a plurality of bolt insertion holes 333a arranged on a virtual circular line centering on the bolt 332 at intervals. The other end of the supporting arm 334 is fixed to a respective mount 333 with a fixing bolt L inserted into one of the bolt insertion holes 333a. In some embodiments, the position of the roller main body 331 can be changed by changing the inclination angle of the supporting arm 334, by selecting a higher or lower bolt insertion hole 333a as shown in FIG. 11. By changing the position of the roller main body 331, the height of the roller main body 331 can be adjusted in accordance with the user's height, etc. By changing the position of the roller main body 331, an ideal ascending angle θ1 may be achieved.

In some embodiments, a movable plate (not shown) is disposed on the mount 333 instead of the cylindrical roller main body 331. In some embodiments, the movable plate is curved. For example, the movable plate may be convex, to better fit a user's bent knees on the plate. Like the cylindrical roller main body 331, the height of the movable plate may be adjusted. Also, the position of the movable plate may also be changed by changing the inclination angle of the supporting arms 334. In other embodiments, the movable plate is flat. In some examples, the movable plate also rotates on an axis and may swing back and forth in an x-direction. In some examples, ends (in the x-direction) of the movable plate have a greater weight than a center of the movable plate, thus inclining the movable plate to move in an x-direction. In some embodiments, the movable plate has a length (in the x-direction) of 12 inches, a width (in the z-direction) of 15 inches, and a height (in the y-direction) of 1.5 inches.

In the stretcher S of this embodiment, as shown in FIG. 11, depending on the user's body length, the pillow member P is positioned by fitting the ridge portion R into one of the grooves G. Then, the user lays down on the floor member 310 in such a manner that the user's waist or hip portion is arranged on the friction reducer 350 and the user's lower leg portion is arranged on the roller main body 331 at a certain downwardly inclined angle. In this embodiment, due to the weight of the lower leg portion of the user A, the roller main body 331 is rotated clockwise, causing a stretching force of the upper leg portion of the user A, which in turn generates a stretching force of the lower body of the user A.

In some embodiments, the roller main body 331 is provided with a pair of right and left dented portions 331a and 331a curved to fit the user's lower leg portion, so as to reduce the pressing force to be applied to the lower leg portion. In some embodiments, the roller main body 331 can include a cushioned surface layer.

During the stretching operation, the upper body of the user A is held in position because of the pillow member P immovably disposed on the base 310 and the increased friction between the upper body of the user and the base 310 due to the existence of the grooves G. In some embodiments, the grooves G may be used both to position the pillow member P and to increase the friction between the upper body of the user and the base 310. The existence of the friction reducer 350 effectively enhances the stretching performance in the same manner as in the aforementioned first to third embodiments.

The stretching operation using this stretcher S is to be performed for a certain period of time. For example, the user may use the stretcher S for at least 15 to 30 minutes. The user may also sleep through the night while in the stretcher S or may remain in the stretcher for a longer period of time. In some embodiments, the stretching operation may include the user laying or sleeping in the stretcher. In some embodiments, the stretching operation may also include the user exercising in the stretcher S for a certain period of time. For example, the user may move his legs back and forth in the x-direction while attached to the stretcher to exercise while using the stretcher S. In other examples, the user may move his lower body in the x-direction on the slidable plate or other friction reducer to exercise while using the stretcher S.

After completion of the stretching operation, the chin strap 40 and the ankle strap 62 are detached from the user A, and the user A may move to the other side of the bed B (i.e. by rolling over), maintaining a laying posture for at least a certain period of time or for an entire night of sleep on the bed B. In some embodiments, the user A may sleep in the stretcher S and may move to another side of the bed B and may maintain a laying posture for a certain amount of time. For example, the user may maintain a laying posture for at least 15 minutes after the stretching operation. In some embodiments, the user may maintain a laying posture for at least 30 minutes after the stretching operation. This effectively prevents joints of the user A from being pressed in the compressing direction even after the stretching operation, resulting in remarkable benefits for treatment. In some embodiments, the stretcher S is arranged on the bed B or arranged adjacent to the bed B with the upper surface of the stretcher S flush with the upper surface of the bed B so that the user A can immediately move on the bed after completion of the stretching operation using the stretcher S.

The angles θ1 and θ2 may be the same or similar to the same heights and angles of the first embodiments. In this embodiment, the angle θ1 may the be angle of the user's upper portion of the legs (i.e. from the knee to the hip) relative to the upper surface of the base 10. Other dimensions discussed with respect to the first embodiment may be the same or similar to respective dimensions of the fourth embodiment.

The other structures are the same as those of the first embodiment, and therefore the explanation will be omitted by allotting the same or corresponding reference numeral to the corresponding portion.

In embodiments of each of the stretchers described herein, an ascending angle θ1 of an upper leg portion of the user A may be the angle of a user's leg during a normal walking stance. By doing so, the user may experience optimal stretching at an angle that may be best for the user's joints. In these embodiments, the angle of the user's leg during a normal walking stance may be the angle of the user's leg after taking a step, relative to a user's leg if it were perpendicular with a flat surface.

In the aforementioned embodiments, it should be noted that the stretching effects can be effectively obtained by positively moving the leg(s) of the user against the stretching force of the stretcher. In some embodiments, the explanations were directed to the case in which the stretchers are used to stretch the lower leg portion including a spine, waist or knee joints in a state in which both leg portions are pulled by the stretching force without moving the leg portions of the user. In other embodiments, in a state in which only one of the legs is pulled by the stretching force, the user can move the leg against the stretching force generated by the stretching force generation mechanism. In some embodiments, it should be noted that the stretcher can be used as an exercise device capable of stretching joints of the lower body of the user during the exercise. The stretcher is not limited to be used by a user having some joint problems, but can be used by a user currently having no joint problems to prevent the occurrence of possible joint problems inherent in a human beings.

The following paragraphs set forth characteristics from three example stretchers, A, B, and C that were prepared and tested.

(Tension of Rubber Cord)

Three stretchers A, B and C according to the first embodiment were prepared in which the height H2 of the cord at the foot side box 30 and the tension of the rubber cords 61 were changed. These stretchers were designed so that a tension of 10 kg (22.05 lb) to 14 kg (30.86 lb) is given to the leg portion of the user during the stretching operation.

If the tension applied to the legs is 10 kg (22.05 lb), the tension applied to one leg will be 5 kg (11.02 lb)). The tension applied to the spine will be less than 5 kg (11.02 lb) because of frictional loss. In an embodiment with the slide plate 50, the tension applied to the spine will be almost the same as the tension applied to the leg portion. If the tension applied to the leg portion is 10 kg (22.05 lb), the actual tension of rubber cords 61 is about 14 kg (30.86 lb) because of the tension loss due to the friction in the pulley system. If a user has trained muscles by sports or the like, the user may stand more than 13 kg of tension.

(Convenient Pulley System)

The ankle straps 62 and rubber cords 61 were connected by the cord via two pulleys 32 and cord guides 33. In some embodiments, the coefficient of friction of the pulley system was about 0.4. In some embodiments, the foot board 30 is made of 13 mm (½″) plywood. In some embodiments, the coefficient of friction of the cord insertion hole 31a was more than 0.6. After attaching the ankle straps 62, although a user has to move the body toward the head side to attach the chin strap 40, the user can move easily without moving against a tension because of the friction between the cord insertion hole 31a and the cord. After attaching the chin strap 40, the user can start stretching by reducing the friction between the cord and the cord insertion hole 31a by raising his or her legs. The user can stop the stretching operation using the friction between the cord and the cord insertion hole 31a by simply lowering the legs. The user can use weight members instead of the rubber cords 61, as shown in FIGS. 6 and 7. In some embodiments, the user may use two 7 kg (15.43 lb) weight members. In some embodiments, weight members are used instead of rubber cords in the stretcher. In these embodiments, it is not necessary to prepare a stretcher depending on the body size of the user because the tension of cord is the same regardless of the body size. In these embodiments, the user can use the stretcher S only on the bed B because it is required to keep a certain height for the up-and-down movements of the weight members. The use of the pulley system in some embodiments may reduce production cost of the stretcher by having the stretcher be simple in structure. If an electric motor is used to control the tension, no pulley system will be required. In embodiments in which an electric motor is used, the stretcher may be configurable by a user, such that the user may control the stretching force to be between 0 kg, 10 kg (22.05 lb) and 5 kg (11.02 lb). For example, the user can select the desired stretching force by operating the switch of the motor, and the stretching operation time can be controlled by a timer function. In these embodiments, one stretcher can be used for different users.

(Pull Legs at 10 Degree Elevation Angle)

The ascending angle θ1 of the upper leg portion may be set to 10 degree (This angle can be changed depending on the user's leg length). This ascending angle can be adjusted by providing a cushion or the like below the user's lower body portion (waist or hip portion).

(How to Stretch Joints)

In some embodiments, the stretcher A is provided with a carpet adhered on the surface of the head side section of the upper surface of the base. The carpet functions as a friction increasing member. In an embodiment in which only one leg is stretched, a single rubber cord 61 may be used.

(Handle Rail)

By holding the grip portions 12, a user may start or stop the stretching operation by raising or lowering the legs. By holding the grip portions 12, the user may move the body in the length direction of the stretcher easily for attaching or detaching the ankle straps 62 and chin strap 40. In embodiments in which the stretcher S is foldable, by holding the grip portions 12, the user may raise the body easily and carry the stretcher easily.

(Slide plate)

Four bearings were provided on the slide plate 50 in order to reduce friction. In some embodiments, the slide plate 50 is effectively disposed under the 4^{th} to 6^{th} sub dorsal part of spinal canals from the hip bone open.

In some embodiments, the stretchers have the following dimensions:

Stretcher A: designed for a bed of 155 cm⁻¹⁷⁵ cm (5.09°-5.74°) length: Length of the base: 190 cm (6.23°), and Height H2 of the cord: 25 cm (0.821°).

Stretcher B: designed for a bed of 175 cm⁻¹⁹⁵ cm (5.74°-6.40°) length: Length of the base: 210 cm (6.89°), Height H2 of the cord: 27 cm (0.89°.)

Stretcher C: designed for a bed of 195 cm-215 cm (6.40°-7.05°) length: Length of the base: 230 cm (7.55°), Height H2 of the cord: 29 cm (0.95°).

In some embodiments, the height H2 of the cord may be about 5-25% of the length of the base. In some embodiments, and as described above, the height H2 may be about 10-15% or even 12-13% of the length of the base.

The tension of the rubber cords differs depending on the body height of the user, so the stretching time is determined based on the body height. In some embodiments, the stretching time is as follows:

If the user is 155 cm (5.09°) in height, the stretcher A is used for 40 minutes (Tension 14 kg (30.86 lb)).

If the user is 165 cm (5.41°) in height, the stretcher A is used for 50 minutes (Tension 12 kg (26.46 lb)).

If the user is 175 cm (5.74°) in height, the stretcher A is used for 60 minutes (Tension 10 kg (22.05 lb)).

If the user is 175 cm (5.74°) in height, the stretcher B is used for 40 minutes (Tension 14 kg (30.86 lb)).

If the user is 185 cm (6.07°) in height, the stretcher B is used for 50 minutes (Tension 12 kg (26.46 lb)).

If the user is 195 cm (6.40°) in height, the stretcher B is used for 60 minutes (Tension 10 kg (22.05 lb)).

If the user is 195 cm (6.40°) in height, the stretcher C is used for 40 minutes (Tension 14 kg (30.86 lb)).

If the user is 205 cm (6.73°) in height, the stretcher C is used for 50 minutes (Tension 12 kg (26.46 lb))

If the user is 215 cm (7.05°) in height, the stretcher C is used for 60 minutes (Tension 10 kg (22.05 lb))

In some embodiments, the attaching height of the left and right ankle straps is set to 20 cm (7.87″). The distance between the fixing members 21 and 21 for fixing the chin strap 40 may be set to 5 cm (0.16°). The length of the grip member 12 may be 60 cm (1.97°). The location of the ankle straps (e.g., ankle straps 62, 162) may be as follows:

In the stretcher A: 60 cm (1.9T) from the foot side box 30

In the stretcher B: 70 cm (2.301) from the foot side box 30

In the stretcher C: 80 cm (2.621) from the foot side box 30

The height of the slide plate 50 with rollers may be set as follows. In some embodiments, the height of the slide plate 50 depends on the length of the user's spinal canals.

In the stretcher A: 5 cm (1.97″)

In the stretcher B: 6 cm (2.36″)

In the stretcher C: 7 cm (2.76″)

The above-disclosed subject matter is to be considered illustrative and not restrictive, and the appended claims are intended to cover all such modifications, enhancements, and other embodiments, which fall within the true spirit and scope of the disclosed embodiments. Thus, the invention is to be construed by the broadest permissible interpretation of the following claims and their equivalents, and shall not be restricted or limited by the foregoing detailed description.

What is claimed is:

1. A stretcher for stretching a lower body of a user, the stretcher comprising:
a base including a first section configured to hold an upper body of the user and a second section adjacent the first section, the second section configured to hold the lower body of the user, the length of the base from a head end to a foot end being L1, wherein the first section is configured to provide a first amount of friction F1 to the body of the user and the second section is configured to provide a second amount of friction F2 to the body of the user, where F2 is less than F1;
an upper body movement restraint disposed at the head end of the base, the upper body movement restraint configured to prevent the upper body of the user from being moved in a longitudinal direction away from the head end of the base when a user is disposed in the stretcher; and
a tension generator disposed at the foot end of the base, the tension generator comprising:
a cord guide attached at the foot end of the base;
at least a first lower body attachment connected via a cord to a cord hole disposed in a front surface of the cord guide, a height between an upper surface of the base and the cord hole being H1,
wherein a first end of the cord is attached to the first lower body attachment, the cord extending from the first lower body attachment to the cord hole and through an inside of the cord guide,
wherein the tension generator is configured to generate a stretching force via the cord to the lower body of the user when a portion of the upper body of the user is disposed in the upper body movement restraint, the lower body of the user is disposed on the second section, and a portion of the lower body of the user is connected to the first lower body attachment, and
wherein the height H1 is 5%-25% of the base length L1.

2. The stretcher of claim 1, wherein the stretcher is integral to a bed.

3. The stretcher of claim 1, wherein the base further comprises a hinge disposed between the first section and the second section, the stretcher being foldable at the hinge.

4. The stretcher of claim 1, wherein the cord extends through an inside of the cord guide via at least a first pulley, the tension generator further comprising at least one of a first rubber cord and a first spring disposed in the base, a second end of the cord attached to the one of the first rubber cord and the first spring.

5. The stretcher of claim 1, wherein the cord extends through a center of the cord guide via at least a first pulley and through a bottom of the cord guide, the tension generator further comprising at least one weight detachably attached to a second end of the cord.

6. The stretcher of claim 1, further comprising a plurality of grooves disposed at an upper surface of the first section of the base.

7. The stretcher of claim 1, further comprising a movable plate disposed on the second section, the movable plate configured to provide the friction F2.

8. The stretcher of claim 1, wherein the upper body movement restraint comprises an adjustable chin strap.

9. The stretcher of claim 1, wherein, when the user is disposed in the stretcher such that a portion of the upper body of the user is disposed in the upper body movement restraint, the lower body of the user is disposed on the second section, and a portion of the lower body of the user is attached to the first lower body attachment, the lower body of the user is disposed at an angle θ1 relative to an upper surface of the base, the angle θ1 being between 8 and 15 degrees.

10. A stretcher for stretching a lower body of a user, the stretcher comprising:
a base including a first section configured to hold an upper body of the user and a second section adjacent the first section, the second section configured to hold the lower body of the user, the length of the base from a head end to a foot end being L1, wherein the first section is configured to provide a first amount of friction F1 to the body of the user and the second section is configured to provide a second amount of friction F2 to the body of the user, where F2 is less than F1;
an upper body movement restraint disposed at the head end of the base, the upper body movement restraint configured to prevent the upper body of the user from being moved in a longitudinal direction away from the head end of the base when a user is disposed in the stretcher; and
a tension generator disposed at the foot end of the base, the tension generator comprising:
a roller support adjacent the foot end of the base that protrudes upwardly;
a roller rotatable about an axis thereof, the roller attached at a top end of the roller support,
wherein the tension generator is configured to cause a stretching force via the roller to the lower body of the user when a portion of the upper body of the user is disposed in the upper body movement restraint, the lower body of the user is disposed on the second portion, and a portion of the lower body of the user is disposed on the roller.

11. The stretcher of claim 10, wherein a height of the roller is adjustable.

12. The stretcher of claim 10, further comprising a plurality of grooves disposed at an upper surface of the first section of the base.

13. The stretcher of claim 12, wherein the upper body movement restraint comprises a pillow, the pillow including one or more ridges, the ridges configured to be disposed in one or more of the plurality of grooves.

14. The stretcher of claim 10, wherein, when the user is disposed in the stretcher such that a portion of the upper body of the user is disposed in the upper body movement restraint, the lower body of the user is disposed on the second section, and a portion of the lower body of the user is disposed on the roller, a portion of the lower body of the user is disposed at an angle θ1 relative to an upper surface of the base, the angle θ1 being between 8 and 15 degrees.

15. A stretcher for stretching a lower body of a user, the stretcher comprising:
a base including a first section configured to hold an upper body of the user and a second section adjacent the first section, the second section configured to hold the lower body of the user, wherein the first section is configured to provide a first amount of friction F1 to the body of the user and the second section is configured to provide a second amount of friction F2 to the body of the user, where F2 is less than F1;
an upper body movement restraint disposed at the head end of the base, the upper body movement restraint configured to prevent the upper body of the user from being moved in a longitudinal direction away from the head end of the base when a user is disposed in the stretcher; and
a tension generator disposed at the foot end of the base, the tension generator comprising:
a cord guide attached at the foot end of the base;
at least a first lower body attachment connected via a cord to a cord hole disposed in a front surface of the cord guide, wherein a first end of the cord is attached to the first lower body attachment, the cord extending from the first lower body attachment to the cord hole and through an inside of the cord guide;
wherein the tension generator is configured to generate a stretching force via the cord to the lower body of the user when a portion of the upper body of the user is disposed in the upper body movement restraint, the lower body of the user is disposed on the second section, and a portion of the lower body of the user is connected to the first lower body attachment, and
wherein the stretcher is integral with a bed.

16. The stretcher of claim 15, wherein the cord extends through an inside of the cord guide via at least a first pulley and through a bottom of the cord guide, the tension generator further comprising at least one weight detachably attached to a second end of the cord.

17. The stretcher of claim 15, wherein the cord extends through an inside of the cord guide via at least a first pulley, the tension generator further comprising at least one of a first rubber cord and a first spring disposed in the base, a second end of the cord attached to the one of the first rubber cord and the first spring.

18. The stretcher of claim 15, wherein the upper body movement restraint comprises a plurality of grooves disposed at an upper surface of the first section of the base.

19. The stretcher of claim 15, wherein the upper body movement restraint comprises an adjustable chin strap.

20. The stretcher of claim 15, wherein, when the user is disposed in the stretcher such that a portion of the upper body of the user is disposed in the upper body movement restraint, the lower body of the user is disposed on the second section, and a portion of the lower body of the user is attached the first lower body attachment, the lower body of the user is disposed at an angle θ1 relative to an upper surface of the base, the angle θ1 being between 8 and 15 degrees.
